(19) United States
(12) Patent Application Publication
(10) Pub. No.: US 2015/0299753 A1
(43) Pub. Date: Oct. 22, 2015

(54) WHOLE GENOME AMPLIFICATION METHOD AND APPLICATION THEREOF

(71) Applicants: Kui WU, Shenzhen (CN); Hanjie WU, Shenzhen (CN); Yong HOU, Shenzhen (CN); Xun XU, Shenzhen (CN); Jun WANG, Shenzhen (CN)

(72) Inventors: Kui WU, Shenzhen (CN); Hanjie WU, Shenzhen (CN); Yong HOU, Shenzhen (CN); Xun XU, Shenzhen (CN); Jun WANG, Shenzhen (CN)

(73) Assignee: BGI TECH SOLUTIONS CO., LTD., Shenzhen (CN)

(21) Appl. No.: 14/378,935

(22) PCT Filed: Mar. 30, 2012
(86) PCT No.: PCT/CN2012/073348

§ 371 Date: Aug. 14, 2014

(51) Int. Cl. C12P 19/34; C12Q 1/68

(52) U.S. Cl. CPC C12P 19/34; C12Q 1/6806

(57) ABSTRACT

Provided are a whole genome sample amplification method, a whole genome sequencing method, and a method for determining whether an abnormal state occurs in a whole genome, a whole genome sample amplification apparatus, a whole genome sequencing device, and a system for determining whether an abnormal state occurs in a whole genome. The whole genome sample amplification method comprises: subjecting a whole genome sample to a first amplification reaction, so as to obtain a first amplification product; and subjecting the first amplification product to a second amplification reaction, so as to obtain a second amplification product. The first amplification reaction is one of the PCR-based amplification reaction and the isothermal amplification reaction, and the second amplification reaction is the other of the PCR-based amplification reaction and the isothermal amplification reaction.

### FIELD

Embodiments of the present disclosure generally relate to whole genome amplification method and application thereof, more particularly, to a method of amplifying a whole genome sample, a method for sequencing a whole genome, a method determining whether an abnormal state occurs in a whole genome, an apparatus of amplifying a whole genome sample, a device of sequencing a whole genome, and a system of determining whether an abnormal state occurs in a whole genome.

### BACKGROUND ART

The statements in this section merely provide background information related to the present disclosure and may not constitute prior art.

Recently, whole genome amplification (WGA) technology is an in-vitro amplification method with limited DNA or single cells to produce enough DNA. Current scientists have co-developed two kinds of strategies to achieve amplification of WGA based on different basic principles, which are PCR-based amplification strategy and isothermal amplification strategy, respectively. The most representative methods comprise Degenerate Oligonucleotide-Primed PCR (DOP-PCR) and Multiple Displacement Amplification (MDA).

However, current technology of whole genome amplification still needs to be improved.

### SUMMARY

Embodiments of the present disclosure seek to solve at least one of the problems existing in the prior art to at least some extent.

The present disclosure is completed based on following discoveries by inventors:

A PCR-based whole genome amplification method, for example, a primer for Degenerate Oligonucleotide-Primed PCR (DOP-PCR) is composed of specific nucleotide sequence at 3′-15′-ends thereof and six random nucleotide sequences in the middle. The PCR procedures thereof are: performing several cycles of low stringency amplification under low annealing temperature; then performing dozens of cycles of stringency amplification under increased annealing temperature. Since the design of DOP-PCR primer at 3′-end is based on the sequence having a high frequent appearance in genome, the designed primer may anneal with genome at several sites under the condition of low stringency amplification initially performed, so as to amplify the genome widespread. Then the product of low stringency amplification is amplified again during the next stringency amplification. Since the DOP-PCR primer has a plurality of annealing sites in the entire genome, the primer and DNA polymerase having an equal quantity may saturate to enter into a linear growth period within the first few cycles. In addition, the inventors find out that, the characteristic of linear growth is particularly benefit for subsequent study on copy number. However, inventors further find out that since DOP-PCR needs pre-fragmentation the genome sample, then ligating adaptor for amplification to the obtained fragment at both ends, by which may generate larger influence on subsequent genome coverage. The inventors find out that using the DOP-PCR method, the current coverage of genome region that can be achieved is only 30% of the maximum.

Comparing with DOP-PCR technology, Multiple Displacement Amplification (MDA) is now widely recognized as the best method for amplifying a whole genome of a single cell. Using a random primer and a template DNA, MDA may bind Phi29 DNA polymerase at a plurality of annealing sites and start replication at the plurality of annealed sites simultaneously. Phi29 DNA polymerase may synthesize DNA along the DNA template, while replacing a complementary strand of the template; the replaced complementary strand of the template then becomes a new template, which is amplified by a randomly combined primer. Phi29 DNA polymerase used by MDA reaction has a strong template-binding capacity for template, which may continuously amplify 10 Kb of DNA templates without disassociation, meanwhile such enzyme also has 3′-5′ exonuclease activity, which may guarantee high fidelity of DNA replication. Thus, a trace of DNA sample may be amplified by MDA to finally obtain a large amount of high quality DNA with high molecular weight, and low level of amplification bias and mutation accumulation. However, inventors of the present disclosure find out that, although MDA technology provides a simple and efficient solution for karyotype analysis, comparison of genomic hybridization and genome sequencing, the inherent characteristic of MDA technology may also cause application bottleneck in some fields. The inventors find out that non-specific background amplification of contamination derived from exogenous DNA or random primer in reacting solution affects the determination of MDA result in concentration detection to a large extent, which needs a PCR result of corresponding species to evaluate MDA efficiency at the same time; in addition, a chimera generated by the amplification characteristic of Phi29 polymerase may cause a great interference to subsequent analysis of copy number variant (CNV) in genome.

According to embodiments of a first broad aspect of the present disclosure, there is provided a method of amplifying a whole genome sample. According to embodiments of the present disclosure, the method may comprise: subjecting the whole genome sample to a first amplification reaction, to obtain a first amplification product; subjecting the first amplification product to a second amplification reaction, to obtain a second amplification product, wherein the first amplification reaction is one of a PCR-based amplification reaction and an isothermal amplification reaction, the second amplification reaction is the other of the PCR-based amplification reaction and the isothermal amplification reaction. The method of amplifying the whole genome sample according to embodiments of the present disclosure may be used to reduce the chimera generated by isothermal amplification reaction and the amplification bias under the premise of ensuring a high coverage of genome. Besides, the inventors also find out that the amplified product obtained using the amplification method of the present disclosure may be used in analyzing copy number variation by chromosome in genome (such as chromosome addition, deletion and transfer). In addition, the amplification method according to embodiments of the present disclosure may be used in simultaneously detecting multiple abnormal states in micro-sample, such as simultaneously detecting single nucleotide polymorphism (SNP) and copy number variation (CNV), to provide more comprehensive information of genome abnormality.

According to embodiments of a second broad aspect of the present disclosure, there is provided a method for sequencing a whole genome. According to embodiments of the present disclosure, the method may comprise: amplifying a whole genome sample according to the method mentioned above, to obtain a whole genome amplified product; constructing a whole genome sequencing-library based on the whole genome amplified product,; and subjecting the whole genome sequencing-library to sequencing. The sequencing result obtained by the method of sequencing the whole genome according to embodiments of the present disclosure, by which the amplified product obtained by specific amplification method is sequenced, may be effectively used in analyzing copy number variation by chromosome in genome (such as chromosome addition, deletion and transfer). Besides, the sequencing result obtained by the sequencing method according to embodiments of the present disclosure, may be used in simultaneously detecting multiple abnormal states in micro-sample, such as simultaneously detecting single nucleotide polymorphism (SNP) and copy number variation (CNV), to provide more comprehensive information of genome abnormality.

According to embodiments of a third broad aspect of the present disclosure, there is provided a method of determining whether an abnormal state occurs in a whole genome. According to embodiments of the present disclosure, the method may comprise: subjecting the whole genome to sequencing according to the method mentioned above, to obtain sequencing data; determining whether the abnormal state occurs in the whole genome based on the sequencing data. The method of determining whether the abnormal state occurs in the whole genome according to embodiments of the present disclosure, based on the whole genome amplified product (which may reflect real state of the whole genome) obtained by the sequencing method according to embodiments of the present disclosure, may effectively analyze copy number variation by chromosome in genome (such as chromosome addition, deletion and transfer), and may simultaneously detect multiple abnormal states in micro-sample, such as simultaneously detect single nucleotide polymorphism (SNP) and copy number variation (CNV), to provide more comprehensive information of genome abnormality.

According to embodiments of a fourth broad aspect of the present disclosure, there is provided an apparatus of amplifying a whole genome sample. According to embodiments of the present disclosure, the apparatus may comprise: a first amplifying unit, suitable for subjecting the whole genome sample to a first amplification reaction, to obtain a first amplification product; a second amplifying unit, connected to the first amplifying unit, suitable for subjecting the first amplification product to a second amplification reaction, to obtain a second amplification product, wherein the first amplifying unit is suitable for performing one of a PCR-based amplification reaction and an isothermal amplification reaction, the second amplifying unit is suitable for performing the other of the PCR-based amplification reaction and the isothermal amplification reaction. The apparatus of amplifying the whole genome sample according to embodiments of the present disclosure may be used to effectively implement the method of amplifying the whole genome sample according to embodiments of the present disclosure, which may be able to reduce the chimera generated by isothermal amplification reaction and the amplification bias under the premise of ensuring a high coverage of genome. Besides, the obtained amplified product may be used in analyzing copy number variation by chromosome in genome (such as chromosome addition, deletion and transfer), and may be also used in simultaneously detecting multiple abnormal states in micro-sample, such as simultaneously detecting single nucleotide polymorphism (SNP) and copy number variation (CNV), to provide more comprehensive information of genome abnormality.

According to embodiments of a fifth broad aspect of the present disclosure, there is provided a device of sequencing a whole genome. According to embodiments of the present disclosure, the device may comprise: a whole genome amplifying apparatus mentioned above; a sequencing-library constructing apparatus, connected to the whole genome amplifying apparatus, suitable for constructing a whole genome sequencing-library for a whole genome amplification product; and a sequencing apparatus, suitable for subjecting the whole genome sequencing-library to sequencing. The device for sequencing the whole genome according to embodiments of the present disclosure, may effectively implement the method for sequencing the whole genome; then the obtained sequencing result of the amplified product obtained using specific amplification method, may be effectively used to analyze copy number variation by chromosome in genome (such as chromosome addition, deletion and transfer). Besides, the obtained sequencing result may also be used in simultaneously detecting multiple abnormal states in micro-sample, such as simultaneously detecting single nucleotide polymorphism (SNP) and copy number variation (CNV), to provide more comprehensive information of genome abnormality.

According to embodiments of a sixth broad aspect of the present disclosure, there is provided a system of determining whether an abnormal state occurs in a whole genome. According to embodiments of the present disclosure, the system may comprise: a whole genome sequencing device mentioned above, for subjecting the whole genome to sequencing, to obtain sequencing data; and an analyzing device, connected to the whole genome sequencing device, suitable for determining whether the abnormal state occurs in the whole genome based on the sequencing data. The system of determining whether the abnormal state occurs in the whole genome according to embodiments of the present disclosure may effectively implement the method of determining whether the abnormal state occurs in the whole genome, by which may effectively analyze copy number variation by chromosome in genome (such as chromosome addition, deletion and transfer), and may also be used in simultaneously detecting multiple abnormal states in micro-sample, such as simultaneously detecting single nucleotide polymorphism (SNP) and copy number variation (CNV), to provide more comprehensive information of genome abnormality.

According to embodiments of a seventh broad aspect of the present disclosure, there is provided a kit for amplifying a whole genome. According to embodiments of the present disclosure, the kit may comprise: a first reagent, for performing one of a PCR-based amplification reaction and an isothermal amplification reaction; and a second reagent, for performing the other of the PCR-based amplification reaction and the isothermal amplification reaction, wherein the first reagent and the second reagent are configured in different containers respectively. The kit for amplifying the whole genome according to embodiments of the present disclosure may be used to effectively implement the method of amplifying the whole genome sample according to embodiments of the present disclosure, to achieve an effective amplification with the whole genome.

Additional aspects and advantages of the present disclosure will be given in part in the following descriptions, become apparent in part from the following descriptions, or be learned from the practice of the present disclosure.

### BRIEF DESCRIPTION OF THE DRAWINGS

These and/or other aspects and advantages of embodiments of the present disclosure will become apparent and more readily appreciated from the following descriptions made with reference to the accompanying drawings, in which:

FIG. 1 is a flow chart showing a method of amplifying a whole genome sample according to an embodiment of the present disclosure;

FIG. 2 is a flow chart showing a method for sequencing a whole genome according to an embodiment of the present disclosure;

FIG. 3 is a flow chart showing a method of determining whether an abnormal state occurs in a whole genome according to an embodiment of the present disclosure;

FIG. 4 is a schematic diagram showing an apparatus of amplifying a whole genome sample according to an embodiment of the present disclosure;

FIG. 5 is a schematic diagram showing a device for sequencing a whole genome according to an embodiment of the present disclosure;

FIG. 6 is a schematic diagram showing a system of determining whether an abnormal state occurs in a whole genome according to an embodiment of the present disclosure;

FIG. 7l is a Circos image showing YH lymphocytes amplified by MDA for 2, 4, 8 and 16 hours according to an embodiment of the present disclosure;

FIG. 8 is a Circos image showing a detection with an influence to amplification bias caused by combination of MDA and DOP-PCR two amplification methods according to an embodiment of the present disclosure;

FIG. 9 is a Circos image showing comparison of amplifying effects using MDA between T21 lymphocytes and YH lymphocytes according to an embodiment of the present disclosure;

FIG. 10 is a Circos image showing a detecting result obtained by amplifying T21 lymphocytes using MDA for different durations along with a subsequent DOP-PCR according to an embodiment of the present disclosure; and

FIG. 11 is an enlarged view of a partial FIG. 10.

### DETAILED DESCRIPTION

Reference will be made in detail to embodiments of the present disclosure. The same or similar elements and the elements having same or similar functions are denoted by like reference numerals throughout the descriptions. The embodiments described herein with reference to drawings are explanatory, illustrative, and used to generally understand the present disclosure. The embodiments shall not be construed to limit the present disclosure.

In addition, terms such as “first” and “second” are used herein for purposes of description and are not intended to indicate or imply relative importance or significance. Therefore, features restricted with “first”, “second” may explicitly or implicitly comprise one or more of the features. Furthermore, in the description of the present disclosure, unless otherwise stated, the term “a plurality of” refers to two or more.

Unless specified or limited otherwise, the terms “connected”, “linked” and variations thereof used herein should be broadly understood, for example, it may be a direct connection, or a detachable connection, or an integral connection; and it may be a mechanical linkage, or may be an electric linkage; and it may connect directly, or may connect indirectly through an intermediary; or may be an internal communication between two elements. For those skilled in the art, the specific meaning of the above-mentioned terms in the present disclosure may be understood in accordance with specific conditions.

The present disclosure is completed based on following discoveries by inventors: A PCR-based whole genome amplification method, for example, a primer for Degenerate Oligonucleotide-Primed PCR (DOP-PCR) is composed of specific nucleotide sequence at 3′-15′-ends thereof and six random nucleotide sequences in the middle. The PCR procedures thereof are: performing several cycles of low stringency amplification under low annealing temperature; then performing dozens of cycles of stringency amplification under increased annealing temperature. Since the design of DOP-PCR primer at 3′-end is based on the sequence having a high frequent appearance in genome, the designed primer may anneal with genome at several sites under the condition of low stringency amplification initially performed, so as to amplify the genome widespread. Then the product of low stringency amplification is amplified again during the next stringency amplification. Since the DOP-PCR primer has a plurality of annealing sites in the entire genome, the primer and DNA polymerase having an equal quantity may saturate to enter into a linear growth period within the first few cycles. In addition, the inventors find out that, the characteristic of linear growth is particularly benefit for subsequent study on copy number. However, inventors further find out that since DOP-PCR needs pre-fragmentation the genome sample, then ligating adaptor for amplification to the obtained fragment at both ends, by which may generate larger influence on subsequent genome coverage. The inventors find out that using the DOP-PCR method, the current coverage of genome region that can be achieved is only 30% of the maximum.

Comparing with DOP-PCR technology, Multiple Displacement Amplification (MDA) is now widely recognized as the best method for amplifying a whole genome of a single cell. Using a random primer and a template DNA, MDA may bind Phi29 DNA polymerase at a plurality of annealing sites and start replication at the plurality of annealed sites simultaneously. Phi29 DNA polymerase may synthesize DNA along the DNA template, while replacing a complementary strand of the template; the replaced complementary strand of the template then becomes a new template, which is amplified by a randomly combined primer. Phi29 DNA polymerase used by MDA reaction has a strong template-binding capacity for template, which may continuously amplify 10 Kb of DNA templates without disassociation, meanwhile such enzyme also has 3′-5′ exonuclease activity, which may guarantee high fidelity of DNA replication. Thus, a trace of DNA sample may be amplified by MDA to finally obtain a large amount of high quality DNA with high molecular weight, and low level of amplification bias and mutation accumulation. However, inventors of the present disclosure find out that, although MDA technology provides a simple and efficient solution for karyotype analysis, comparison of genomic hybridization and genome sequencing, the inherent characteristic of MDA technology may also cause application bottleneck in some fields. The inventors find out that non-specific background amplification of contamination derived from exogenous DNA or random primer in reacting solution affects the determination of MDA result in concentration detection to a large extent, which needs a PCR result of corresponding species to evaluate MDA efficiency at the same time; in addition, a chimera generated by the amplification characteristic of Phi29 polymerase may cause a great interference to subsequent analysis of copy number variant (CNV) in genome.

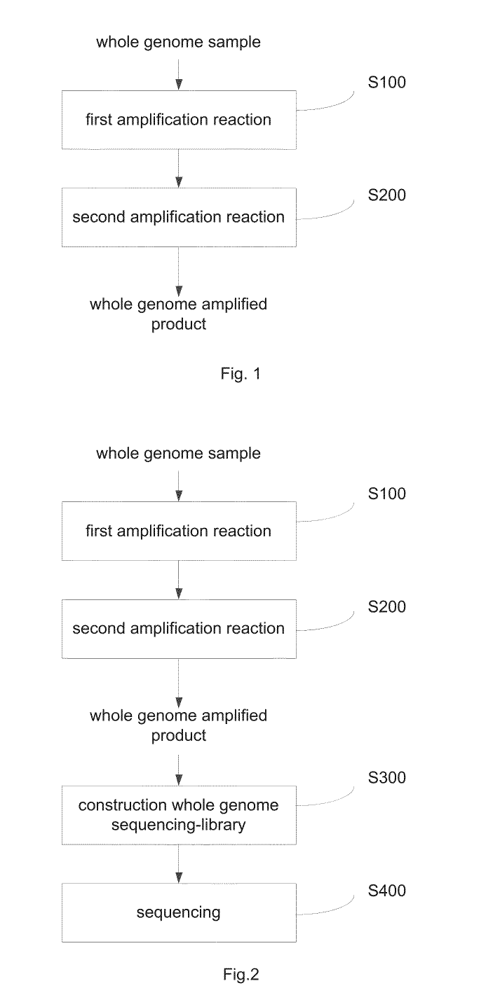

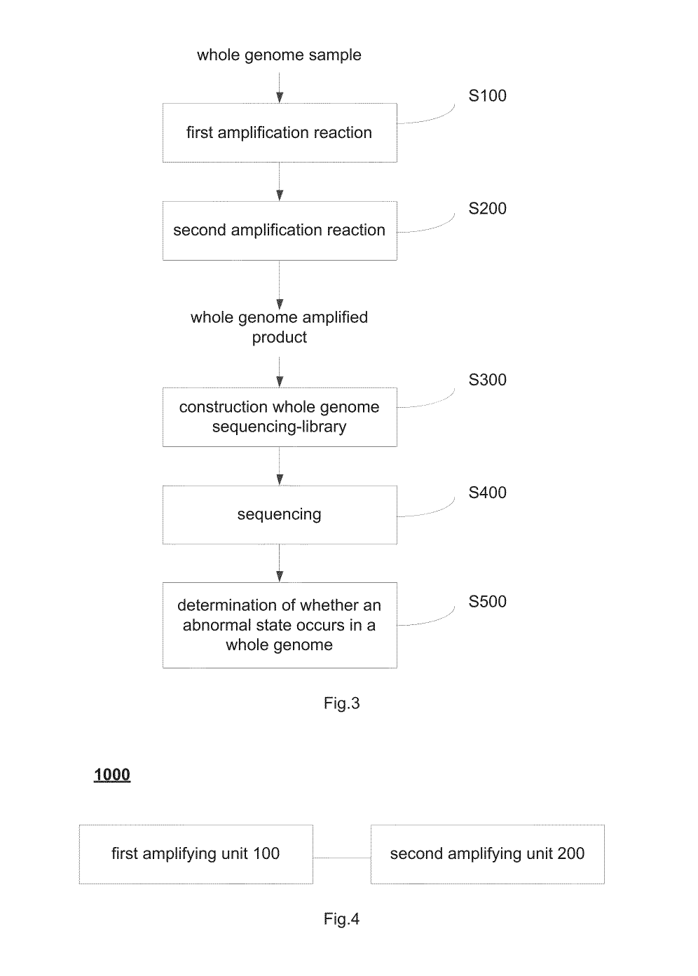

Referring to FIG. 1, a method of amplifying a whole genome sample according to embodiments of the present disclosure is described below. According to embodiments of the present disclosure, the method comprises:

S100: the whole genome sample is subjected to a first amplification reaction, to obtain a first amplification product.

S200: after being obtained, the first amplification product is subjected to a second amplification reaction, to obtain a second amplification product, in which the second amplification product may constitute an amplified whole genome.

According to embodiments of the present disclosure, both the first amplification reaction and the second amplification reaction are selected from one of a PCR-based amplification reaction and an isothermal amplification reaction. Therein, types of the first amplification reaction and the second amplification reaction are different, the first amplification reaction is one selected from the PCR-based amplification reaction and the isothermal amplification reaction, while the second amplification reaction is the other one selected from the PCR-based amplification reaction and the isothermal amplification reaction, for example, the first amplification reaction is the PCR-based amplification reaction, while the second amplification reaction is the isothermal amplification reaction. Accordingly, the method of amplifying the whole genome sample according to embodiments of the present disclosure may be used to reduce the chimera generated by isothermal amplification reaction and the amplification bias under the premise of ensuring a high coverage of genome. Besides, the inventors also find out that the amplified product obtained using the amplification method of the present disclosure may be used in analyzing copy number variation by chromosome in genome (such as chromosome addition, deletion and transfer). In addition, the amplification method according to embodiments of the present disclosure may be used in simultaneously detecting multiple abnormal states in micro-sample, such as simultaneously detecting single nucleotide polymorphism (SNP) and copy number variation (CNV), to provide more comprehensive information of genome abnormality.

The specific type of the term “PCR-based amplification reaction” used herein is not subjected to special restrictions, according to embodiments of the present disclosure, which may be at least one selected from a group consisting of interspersed repetitive sequence (IRS) PCR (the repetitive sequence is Alu repetition), linker adapter technique PCR (LA-PCR), degenerate oligonucleotide-primed PCR (DOP-PCR), primer extension pre-amplification PCR (PEP-PCR), DOP-PCR followed by improved PEP (IPEP-PCR) (long products from low DNA quantities DOP-PCR (LL-DOP-PCR), improved primer extension pre-amplification PCR, ligation-mediated PCR (LMP) (a. single-cell comparative genomic hybridization PCR (SCOMP PCR); b. adaptor-ligation PCR of randomly sheared genomic DNA (PRSG PCR)), and Ominplex amplification. According to embodiments of the present disclosure, DOP-PCR is preferred for the PCR-based amplification reaction. The amplification of DOP-PCR is achieved depending on a set of oligonucleotides having a random sequence at 3′-end and a partially fixed sequence at 5′-end. Such primers are designed to be able to relatively evenly anneal and bind into DNA sample. When the oligonucleotides are bind into the fixed sequence, these products may be subjected to extension and amplification by polymerase. According to embodiments of the present disclosure, DOP-PCR may be achieved using a commercial kit, for example GenomePlex Single Cell Whole Genome Amplification Kit from Sigma Company.

According to embodiments of the present disclosure, the term “isothermal amplification reaction” used herein may also be known as non-PCR-based linear amplification, the specific type thereof is not subjected to special restrictions. According to embodiments of the present disclosure, the isothermal amplification reaction may be at least one selected from a group consisting of strand displacement amplification (SDA), multiple displacement amplification (MDA), and T7-based linear amplification. According to embodiments of the present disclosure, MDA reaction is preferred, i.e. MDA is used as the isothermal amplification reaction. MDA performs isothermal DNA amplification by means of mesophilic DNA polymerase (herein abbreviated as Phi29 enzyme) cloned from Bacillus phage phi29 of Bacillus subtilis and random oligonucleotides primer having six bases with an anti-exonuclease activity. Since the Phi enzyme has a characteristic of strand displacement, the method of amplifying the whole genome is named as multiple displacement amplification (MDA). MDA technology is that: a random primer is used to anneal with a template DNA at a plurality of sites, and Phi29 DNA polymerase starts replication at the plurality of annealed sites simultaneously. Phi29 DNA polymerase synthesizes DNA along the DNA template, while replacing a complementary strand of the template; the replaced complementary strand of the template then becomes a new template, which is amplified by a randomly combined primer. According to embodiments of the present disclosure, MDA amplification reaction may be completed by following procedures: incubating under an isothermal condition at 30° C. for 16 hours in an amplification system containing a genome of a cell, then after heating to 65° C. for 10 minutes, terminating the amplification reaction. MDA amplification may be achieved using a commercial kit, for example using REPLI-g Mini Kit from Qiagen Company.

According to embodiments of the present disclosure, the specific type of the whole genome sample which may be used for the amplification method according to embodiments of the present disclosure is not subjected to special restrictions. The amplification method according to embodiments of the present disclosure, may be effectively amplify a trace of the whole genome sample. As a result, according to embodiments of the present disclosure, the used whole genome sample is a single cell-derived whole genome sample.

According to embodiments of the present disclosure, a sequence of the isothermal amplification reaction and PCR-based amplification reaction is not subjected to special restriction. According to a specific example of the present disclosure, the first amplification reaction is the isothermal amplification reaction, while the second amplification reaction is the PCR-based amplification reaction, i.e. the isothermal amplification reaction is firstly performed, and then the amplified product obtained by the isothermal amplification reaction is subjected to the PCR-based amplification reaction. According to some examples of the present disclosure, the first amplification reaction may be at least one selected from a group consisting of SDA, MDA, and RCA; the second amplification reaction may be at least one selected from a group consisting of LA-PCR, DOP-PCR, PEP, and LA-PCR. According to specific embodiments of the present disclosure, MDA is firstly performed, and then the amplified product obtained by MDA is subjected to DOP-PCR, i.e. the first amplification reaction is MDA, while the second amplification reaction is DOP-PCR. According to embodiments of the present disclosure, durations of the first amplification reaction and the second amplification reaction are not subjected to special restriction. According to specific examples of the present disclosure, the first amplification reaction may be performed for 15 minutes to 120 minutes, preferably for 60 minutes to 120 minutes, which may further improve the effect for amplifying the whole genome sample.

In a second aspect of the present disclosure, there is provided a method for sequencing a whole genome. Referring to FIG. 2, according to embodiments of the present disclosure, the method comprises:

firstly, according to the above-mentioned method, amplifying the whole genome sample to obtain a whole genome amplified product.

According to embodiments of the present disclosure, the method may further comprise a step of extracting the whole genome sample from a single cell, and optionally comprises a sub-step of isolating the single cell from a biological sample, which may effectively obtain sequence information of the whole genome of the single cell isolated from the biological sample. According to embodiments of the present disclosure, type of the whole genome extracted from the single cell is not subjected to special restrictions. According to embodiments of the present disclosure, type of the biological sample being as a resource of the whole genome sample is not subjected to special restrictions. According to specific examples of the present disclosure, the used biological sample may be at least one selected from a group consisting of blood, urine, saliva, tissue, germ cell, blastomere and embryo, which may be obtained conveniently from organisms; and may use different samples specific for some certain diseases, so as to use a certain analyzing method for the some certain diseases. According to an embodiment of the present disclosure, the step of isolating the single cell from the biological sample is performed by at least one selected from a group consisting of dilution, mouth-controlled pipette isolation, micromanipulation, flow cytometry isolation and microfluidic, which may effectively and conveniently obtain the single cell of the biological sample, to implement subsequent operations. Optionally, according to embodiments of the present disclosure, the step of extracting the whole genome sample from the single cell may further comprise another sub-step of lysing the single cell, to release the whole genome of the single cell. According to some examples of the present disclosure, methods for lysing the single cell to release the whole genome are not subjected to special restrictions, as long as the single cell can be sufficiently lysed. According to specific example of the present disclosure, an alkaline lysis buffer may be used for lysing the single cell to release the whole genome of the single cell. The inventors find out that, the single cell may be effectively lysed to release the whole genome, and the released whole genome may improve the accuracy when being sequenced, so as to further improve the efficiency of determining chromosome aneuploidy of the single cell.

S300: secondly, constructing a whole genome sequencing-library based on the whole genome amplified product,

S400: subjecting the whole genome sequencing-library to sequencing, which may effectively obtain whole genome information of the single cell, so as to further improve the efficiency of determining chromosome aneuploidy of the single cell.

According to embodiments of the present disclosure, the whole genome sequencing-library is sequenced using at least one selected from a group consisting of Illumina Hiseq2000, SOLiD 454, and single-molecule sequencing apparatus. Those skilled in the art may select different methods of constructing a whole genome sequencing-library in accordance with specific solution of whole-genome sequencing. Details of constructing the whole genome sequencing-library may refer to a specification provided by sequencing-instrument manufacturer, such as Illumina company, for example Multiplexing Sample Preparation Guide (Part#1005361; February 2010) or Paired-End SamplePrep Guide (Part#1005063; February 2010) is referred, which are both incorporated herein by reference.

The sequencing result obtained by the method of sequencing the whole genome according to embodiments of the present disclosure, by which the amplified product obtained by specific amplification method is sequenced, may be effectively used in analyzing copy number variation by chromosome in genome (such as chromosome addition, deletion and transfer). Besides, the sequencing result obtained by the sequencing method according to embodiments of the present disclosure, may be used in simultaneously detecting multiple abnormal states in micro-sample, such as simultaneously detecting single nucleotide polymorphism (SNP) and copy number variation (CNV), to provide more comprehensive information of genome abnormality.

As a result, in a third aspect of the present disclosure, there is provided a method of determining whether an abnormal state occurs in a whole genome. Referring to FIG. 4, according to embodiments of the present disclosure, the method comprises:

subjecting the whole genome to sequencing according to the method mentioned above, to obtain sequencing data; and

S500: determining whether the abnormal state occurs in the whole genome based on the sequencing data.

The method of determining whether the abnormal state occurs in the whole genome according to embodiments of the present disclosure, based on the whole genome amplified product (which may reflect real state of the whole genome) obtained by the sequencing method according to embodiments of the present disclosure, may effectively analyze copy number variation by chromosome in genome (such as chromosome addition, deletion and transfer), and may simultaneously detect multiple abnormal states in micro-sample, such as simultaneously detect single nucleotide polymorphism (SNP) and copy number variation (CNV), to provide more comprehensive information of genome abnormality. According to embodiments of the present disclosure, methods of determining an abnormal state by analyzing the sequencing data are not subjected to special restrictions. According to embodiments of the present disclosure, a method of making a genome Circos image based on sequencing data may be used to determining whether an abnormal state occurs in a whole genome. According to embodiments of the present disclosure, type of the abnormal state is not subjected to special restrictions, which may be at least one selected from a group consisting of SNP and CNV. Details of making a genome Circos image may refer to a tutorial provided by the official website of Circos http://circos.ca/guide/genomic/. The method of analyzing the genome data by making Circos image has been broadly used, in short, using Circos software of version v0.55-1 updated on Jun. 16, 2001, brief steps of making genome Circos image are shown as below:

1. dividing a genome into n regions in accordance with a certain size as required (windows having a size of 10K and 100K respectively are used in the embodiments of the present disclosoure), calculating desired values of each region (such as genome content, GC content in sequence, gene number and etc.), generating data files requied;

2. configuring profiles of Circos as required, which may configure image color, font, size, type (scattergram, bar chart, graphs, heatmap, and etc.), input data file and etc.

3. running Circos, to obtain a genome Circos image.

In a fourth aspect of the present disclosure, there is provided an apparatus of amplifying a whole genome sample. Referring to FIG. 4, according to embodiments of the present disclosure, the apparatus 1000 comprises: a first amplifying unit 100 and a second amplifying unit 200, in which the first amplifying unit 100 is suitable for subjecting the whole genome sample to a first amplification reaction, to obtain a first amplification product; the second amplifying unit 200, connected to the first amplifying unit 100, is suitable for subjecting the first amplification product to a second amplification reaction, to obtain a second amplification product, in which the first amplifying unit 100 is suitable for performing one of a PCR-based amplification reaction and an isothermal amplification reaction, the second amplifying unit 200 is suitable for performing the other of the PCR-based amplification reaction and the isothermal amplification reaction. According to embodiments of the present disclosure, the first amplifying unit 100 is suitable for performing an isothermal amplification reaction, while the second amplifying unit 200 is suitable for performing PCR-based amplification reaction. According to specific examples of the present disclosure, the first amplifying unit 100 is suitable for performing MDA, while the second amplifying unit 200 is suitable for performing DOP-PCR.

Thus, the apparatus 1000 of amplifying a whole genome sample according to embodiments of the present disclosure may be used to effectively implement the method of amplifying a whole genome sample according to the embodiments of the present disclosure, so as to reduce the chimera generated by isothermal amplification reaction and the amplification bias under the premise of ensuring a high coverage of genome. Besides, the obtained amplified product may be used in analyzing copy number variation by chromosome in genome (such as chromosome addition, deletion and transfer), and may be also used in simultaneously detecting multiple abnormal states in micro-sample, such as simultaneously detecting single nucleotide polymorphism (SNP) and copy number variation (CNV), to provide more comprehensive information of genome abnormality. It would be appreciated by those skilled in the art that the above-described features and advantages regarding the amplification method may also be suitable for the apparatus 1000 of amplifying a whole genome sample, so a detailed description thereof will be omitted here.

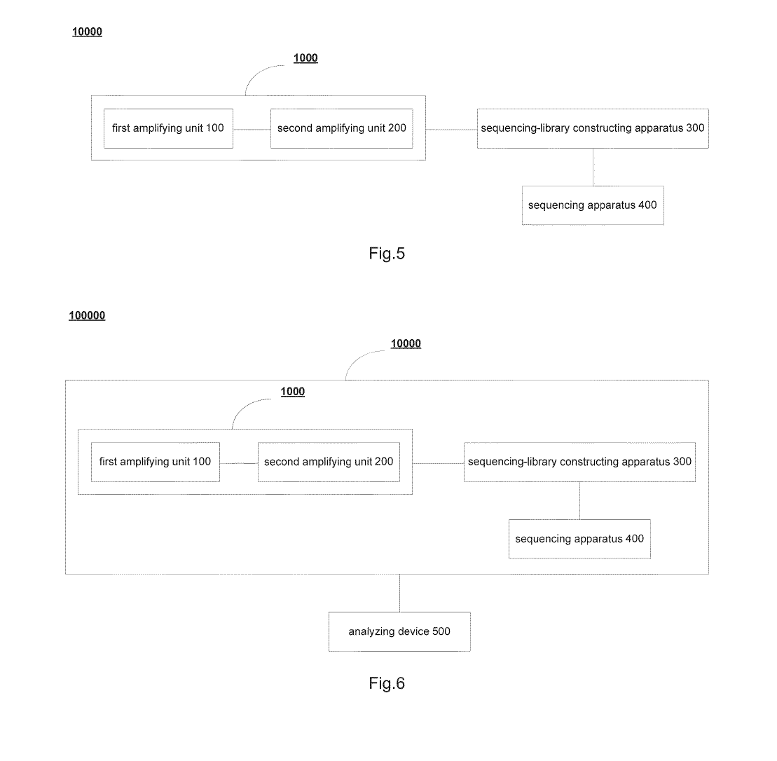

In a fifth aspect of the present disclosure, there is provided a device of sequencing a whole genome. Referring to FIG. 5, according to embodiments of the present disclosure, the device 10000 comprises: a whole genome amplifying apparatus 1000, a sequencing-library constructing apparatus 300, a sequencing apparatus 400.

According to embodiments of the present disclosure, the whole genome amplifying apparatus 1000 has been mentioned above. According to embodiments of the present disclosure, the whole genome amplifying apparatus 1000 further comprises: a single cell isolating unit and a single cell lysing unit, in which the single cell isolating unit is used for isolating the single cell from a biological sample, while the single cell lysing unit is used for receiving and lysing an isolated single cell, to release the whole genome from the single cell. According to specific examples of the present disclosure, the single cell isolating unit may comprise at least one instrument suitable for conducting following operations: dilution, mouth-controlled pipette isolation, micromanipulation, flow cytometry isolation, and microfluidic.

According to embodiments of the present disclosure, the sequencing-library constructing apparatus 300, connected to the whole genome amplifying apparatus 1000, is suitable for constructing a whole genome sequencing-library for a whole genome amplification product; the sequencing apparatus 400 is suitable for subjecting the whole genome sequencing-library to sequencing. According to embodiments of the present disclosure, the sequencing apparatus comprises at least one of selected from a group consisting of Illumina Hiseq2000, SOLiD 454, and single-molecule sequencing apparatus.

Thus, the device for sequencing a whole genome according to embodiments of the present disclosure, may effectively implement the method for sequencing a whole genome; then the obtained sequencing result of the amplified product obtained using specific amplification method, may be effectively used to analyze copy number variation by chromosome in genome (such as chromosome addition, deletion and transfer). Besides, the obtained sequencing result may also be used in simultaneously detecting multiple abnormal states in micro-sample, such as simultaneously detecting single nucleotide polymorphism (SNP) and copy number variation (CNV), to provide more comprehensive information of genome abnormality. It would be appreciated by those skilled in the art that the above-described features and advantages regarding sequencing the whole genome may also be suitable for the device for sequencing the whole genome, so a detailed description thereof will be omitted here.

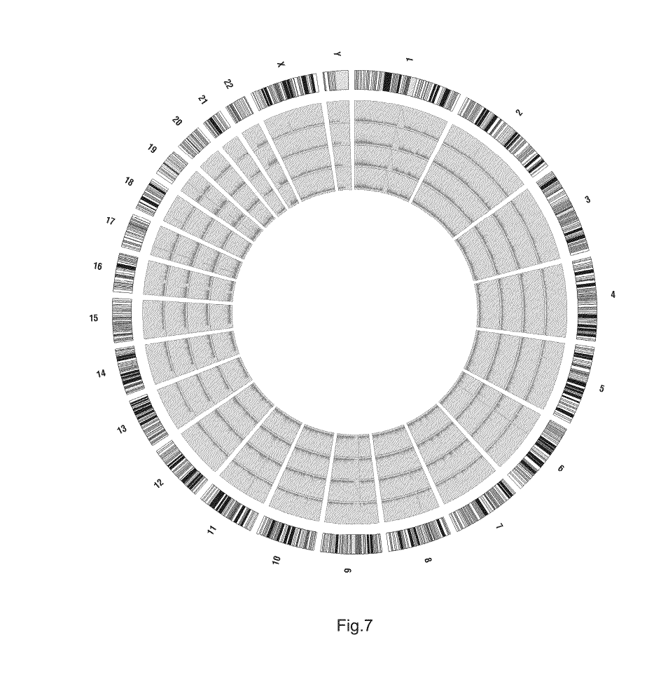

Furthermore, in a sixth aspect of the present disclosure, there is provided a system of determining whether an abnormal state occurs in a whole genome. Referring to FIG. 6, according to embodiments of the present disclosure, the system 100000 comprises: a whole genome sequencing device 10000 and an analyzing device 500. According to embodiments of the present disclosure, the whole genome sequencing device 10000 has been described above, for subjecting the whole genome to sequencing, to obtain sequencing data. According to embodiments of the present disclosure, the analyzing device 500, connected to the whole genome sequencing device 10000, is suitable for determining whether the abnormal state occurs in the whole genome based on the sequencing data. According to embodiments of the present disclosure, type of the analyzing device 500 is not subjected to special restrictions. According to specific examples of the present disclosure, method of making a genome Circos image based on sequencing data may be used to determining whether an abnormal state occurs in a whole genome.

The system of determining whether the abnormal state occurs in the whole genome according to embodiments of the present disclosure may effectively implement the method of determining whether the abnormal state occurs in the whole genome, by which may effectively analyze copy number variation by chromosome in genome (such as chromosome addition, deletion and transfer), and may also be used in simultaneously detecting multiple abnormal states in micro-sample, such as simultaneously detecting single nucleotide polymorphism (SNP) and copy number variation (CNV), to provide more comprehensive information of genome abnormality. It would be appreciated by those skilled in the art that the above-described features and advantages regarding the method of determining whether the abnormal state occurs in the whole genome are still suitable for the system of determining whether the abnormal state occurs in the whole genome, so a detailed description thereof will be omitted here.

In a seventh aspect of the present disclosure, there is provided a kit. According to embodiments of the present disclosure, the kit comprises: a first reagent, for performing one of a PCR-based amplification reaction and an isothermal amplification reaction; and a second reagent, for performing the other of the PCR-based amplification reaction and the isothermal amplification reaction, in which the first reagent and the second reagent are configured in different containers respectively. The kit for amplifying the whole genome according to embodiments of the present disclosure may be used to effectively implement the method of amplifying the whole genome sample according to embodiments of the present disclosure, to achieve an effective amplification with the whole genome.

It should be noted that the method of amplifying a whole genome sample according to embodiments of the present disclosure is completed by inventors of the present disclosure through extremely hard and optimized creative works.

Reference will be made in detail to examples of the present disclosure. It would be appreciated by those skilled in the art that the following examples are explanatory, and cannot be construed to limit the scope of the present disclosure. If the specific technology or conditions are not specified in the examples, a step will be performed in accordance with the techniques or conditions described in the literature in the art (for example, referring to J. Sambrook, et al. (translated by Huang PT), Molecular Cloning: A Laboratory Manual, 3rd Ed., Science Press) or in accordance with the product instructions. If the manufacturers of reagents or instruments are not specified, the reagents or instruments may be commercially available, for example, from Illumina Company.

### EXAMPLE 1

1.1 Single Cell Whole Genome Sequencing

The first Asian sequence from donor “YH” issued in 2008 was used, and a lymphocytic line derived from an Asian healthy male donor was collected as a material of a single cell. Firstly, well-growing lymphocytes was added with an appropriate amount of trypsin to lyse the lymphocytes attached to a culturing dish; then the lysing reaction was terminated by adding a medium containing FBS; the obtained medium containing the desired lymphocytes was collected. By the decontamination method of high-speed centrifuging and removing supernatant, the obtained cell pellet contained in the medium was washed with a PBS solution, and then the washed cell pellet was re-suspended with an appropriate amount of the PBS solution. After transferred to a new culturing dish, the obtained cell suspension was subjected to cell isolation by means of mouth-controlled pipette under an inverted microscope. Then the isolated cells were subjected to amplification method according to the method shown in Table 1.

TABLE 1
Sample ID of YH lymphocyte and specific treatment thereof

| Sample ID | Treatment |
|---|---|
| DOP-2.1 | DOP-PCR amplification |
| MDA16-2.4 | MDA amplification for 16 hours |
| MDA1-DOP-2.2 | MDA amplification for 1 hour |
| | followed by DOP-PCR |
| MDA2-DOP-2.3 | MDA amplification for 2 hour |
| | followed by DOP-PCR |

Specific procedure of DOP-PCR reaction comprised: after collected, the cells were added with Single Cell Lysis & Fragmentation Buffer containing proteinase K, to lyse the cell and release a genome, and then the released genome was subjected to fragmentation to obtain nucleic acid fragments. Subsequently, Single Cell Library Preparation Buffer, Library Stabilization Solution and corresponding enzymes (all from a commercial kit: GenomePlex® Single Cell Whole Genome Amplification Kit) were added into the obtained nucleic acid fragments, to form a first reaction system; and then the reaction system was placed in a thermal cycler and incubated as follows:

16° C. for 20 minutes

24° C. for 20 minutes

37° C. for 20 minutes

75° C. for 5 minutes

4° C. hold.

Then the obtained amplified product was added to Amplification Master Mix and Whole Genome Amplification DNA Polymerase (all from a commercial kit: GenomePlex

Single Cell Whole Genome Amplification Kit of Sigma Company), to obtain a second reaction system; and then the second reaction system was placed in the thermal cycler and incubated as follows:

| | | |
|---|---|---|
| 95° C. for 3 minutes | | |
| 94° C. for 30 seconds | | |
| | {close oversize brace} | 25 cycles |
| 65° C. for 5 minutes | | |
| 4° C. hold. | | |

After the amplification reaction was completed, the obtained DNA product could be directly used for downstream application or stored at −20° C.

In addition, if the sample was subjected to whole genome amplification using MDA, REPLI-g Mini Kit purchased from Qiagen Company was utilized. In short: alkaline lysis buffer (ALB) containing KOH was used to lyse the cell; nucleic acid denaturation buffer prepared using DLB buffer (from a commercial kit: REPLI-g Mini Kit) was added into the lysed cell allowing denaturation at room temperature for 3 minutes, and then a stop solution was added to terminate the denaturation reaction. After added with an amplification buffer containing Phi29 polymerase, the denatured sample was incubated under an isothermal condition at 30° C. for 16 hours, which followed by an incubation under an isothermal condition at 65° C. for 10 minutes to inactivate the polymerase for terminating the amplification reaction. After the amplification reaction was completed, the obtained DNA product could be directly used for downstream application or stored at −20° C. Subsequently, the amplified genome product obtained in accordance with different amplification methods was subjected to sequencing-library constructing according to the method of constructing a short fragment inserted library provided by the manufacturer of Illumina Hiseq2000 platform. In short, the method comprised:

The obtained DNA product was fragmented using Covaris ultrasonic instrument to obtain desired inserted fragments; the obtained fragments were subjected to end-repairing, adding base A to end-repaired DNA, and ligating an adaptor suitable for Pair-end standard and general flowcell of Illumina sequencing platform; then the obtained product ligated with the adaptor was subjected to 10 cycles of amplification with a primer having an Index. Subsequently, the amplified product was subjected to gel electrophoresis for purification; and the target fragment having a certain length of DNA was selected by gel-cutting and collected according to library concentration. The sequencing reaction of a plurality of sequencing-libraries was realized with one lane on one piece of flowcell; and then the obtained sequencing data was subjected to distinguishing in accordance with respectively added index after data generated, so as to obtain sequencing data of each sample.

After the sequencing data was obtained, the raw off-computer data (fastq. file) was subjected to preliminary processing, to remove contaminated data, low quality data and adaptor, to obtain a filtered sequencing data; and then the filtered sequencing data was subjected to sequence-assembling by inputting into SOAP software, to obtain a sequencing depth and coverage of the genome sample. The results thereof were shown as below in Table2.

TABLE 2
sequencing data of YH lymphocyte
obtained using DOP-PCR and MDA

| | Average | Average | | |
|---|---|---|---|---|
| | sequencing | sequencing | | |
| | depth of | depth of | | Median of |
| | covered | whole | | sequencing |
| Sample ID | region | genome | Coverage | depth |
| DOP-2.1 | 5.9062537 | 0.039572376 | 0.67% | 1 |
| MDA16-2.4 | 1.517538109 | 0.188808921 | 12.44% | 1 |
| MDA1-DOP-2.2 | 2.113327153 | 0.857151138 | 40.56% | 1 |
| MDA2-DOP-2.3 | 2.497788155 | 1.07744229 | 43.14% | 2 |

The term “average sequencing depth of covered region” used herein represented a depth value of a genome sequence covered by sequencing data having a length being not less than that of the filtered sequencing data; the term “average sequencing depth of whole genome” used herein represented a ratio between a size of the aligned genome sequence (which was not always able to cover the whole genome region of the species) and a size of the whole species genome; the term “coverage” represented a percentage of a genome region covered by sequencing data having a length being not less than that of the filtered sequencing data to the whole genome; the term “median of sequencing depth” represented a depth value of reads ranked in the middle of all reads which were ranked in accordance with sequencing depth thereof from high to low.

As can be seen from Table2, MDA combining with DOP-PCR were used to amplify YH single cell whole genome (Sample MDA1-DOP-2.2 and MDA2-DOP-2.3), the obtained value of the whole genome average sequencing depth and coverage thereof were obviously higher than the value of genome amplification obtained using DOP-PCR (Sample DOP-2.1) or MDA (Sample MDA16-2.4) along.

1.2 Amplification effect comparison of the whole genome MDA and DOP-PCR in a level of single cell

As described in 1.1, the single lymphocyte derived from YH lymphocytic line was subjected to a whole genome amplification method respectively using MDA and DOP-PCR by the inventors of the present disclosure. PE100 sequencing-library was subjected to sequencing with 0.1X data volume, the obtained results thereof shown in Table3 below:

TABLE 3
Amplification effect comparison between
MDA and DOP-PCR with the single cell

| Sample ID | DOP-2.1 | MDA16-2.4 |
|---|---|---|
| Average sequencing depth | 5.9062537 | 1.517538109 |
| of covered region | | |
| Average sequencing depth | 0.039572376 | 0.188808921 |
| of whole genome | | |
| Coverage | 0.67% | 12.44% |
| Median of sequencing depth | 1 | 1 |

Therefore, MDA method had an obvious advantage in genome coverage, while DOP-PCR method lost a large part of genome information. In addition, MDA method caused non-specific amplification and chimera formation with the random primers, while

DOP-PCR method had a disadvantage of insufficient genome coverage and shorter length of the amplified product.

### EXAMPLE 2

### Whole Genome Amplification Method Capable of Reducing Amplification Bias

For reducing the amplification bias introduced by MDA method, the inventors of the present disclosure decreased the reaction duration of MDA method. A reaction duration of an ordinary MDA method was 16 hours, a reaction duration of a common MDA method had four temperature gradients, which were 2 hours, 4 hours, 8 hours and 16 hours respectively. After obtained from YH lymphocytic line, the single cell was subjected to amplification in accordance with the above described reaction durations of MDA method by the inventors of the present disclosure; and after constructed with the amplified product, a whole genome double ends library was subjected to sequencing. Then the obtained sequencing data was subjected to a method of making a genome Circos image; the obtained cell genome Circos image was shown in FIG. 7. As shown in FIG. 7, the Circos image totally had 5 circles, in which the outermost represented chromosome karyotype information, 4 inner circles respectively showed YH lymphocytes amplified by MDA for 2, 4, 8 and 16 hours from the outside to the inside. As can be seen from FIG. 7, with increasing amplification duration of MDA, a difference of coverage amplified from genome would increase.

The inventors of the present disclosure found out that, an occurring amplification difference was resulted from amplification characteristic of MDA. The random primer in the reaction buffer randomly bound into the template strand, the number of primers bound with allele or different genome site was not always equal, the number difference of the amplified product would increase gradually after a long time amplification. Meanwhile, a difference of GC content in genome would also cause a certain influence to the binding between the random primer and template.

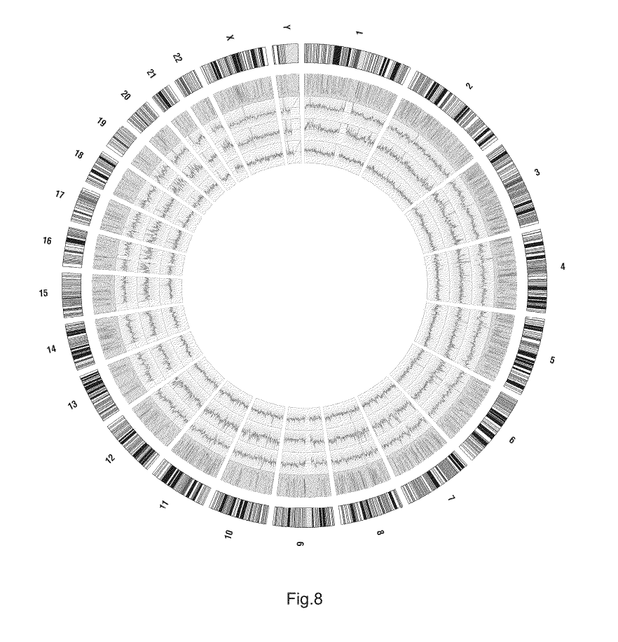

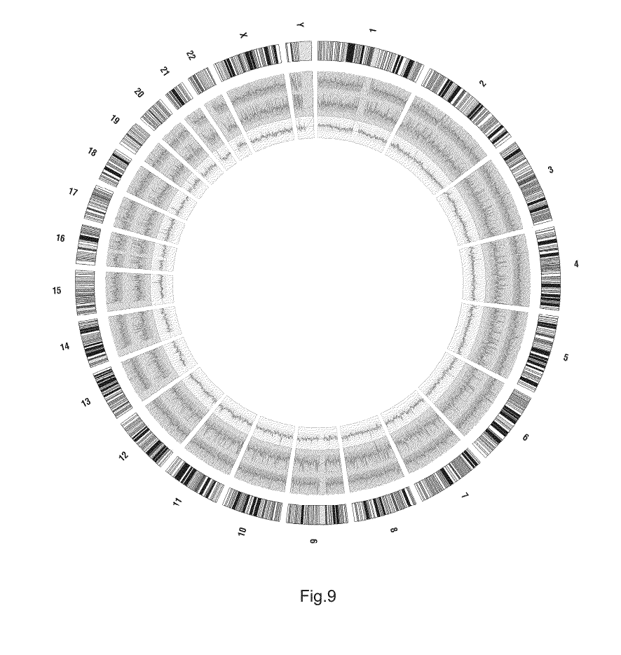

Subsequently, the single cell derived from YH lymphocytes was subjected to amplification method of DOP-PCR and MDA combined with DOP-MDA respectively, and the amplification bias thereof was compared. FIG. 8 was a Circos image shown a detection with an influence to amplification bias caused by combination of MDA and DOP-PCR two amplification methods. As shown in FIG. 8, the circles in the Circos image respectively represented the coverage of genome sample amplified by 4 kinds of amplification solutions, which were DOP-PCR amplification, MDA amplification for 1 hour followed by DOP-PCR, MDA amplification for 2 hour followed by DOP-PCR and MDA amplification for 16 hours, from the outside to the inside. It could be seen from FIG. 8 that a difference of amplification times among sites was relative larger in the circle represented MDA amplification for 16 hours. Due to the specific amplification method of DOP-PCR, the replicated sequences were enriched only in a small number of regions, which caused a rapidly increasing coverage of the region having a relative large difference when compared with an adjacent region. Two middle circles in the Circos image, represented MDA amplification for a period of time followed by DOP-PCR, indicated that the amplification bias of sequence could be reduced under the premise of ensuring a high coverage, which was benefit to CNV analysis of single cell. The amplification method of MDA combined with DOP-PCR would be subsequently investigated in detail.

### EXAMPLE 3

A new verifying method of reducing amplification bias using a somatic cell derived from a patient having Trisomy 21 (T21)

In order to verify that a large range of CNV in genome could be detected by the method of MDA combined whit DOP-PCR, a single cell was subjected to detection of amplification, which derived from peripheral blood lymphocyte that was collected from a donor having Trisomy 21. Firstly, the single cell was subjected to the whole genome amplification, which derived from lymphocytes that was collected from donors of T21 or YH respectively, and the genome Circos images of each sample were obtained according to the above-described method, which were shown in FIG. 9. As shown in FIG. 9, the Circos image showing comparison of amplifying effects with 3 circles respectively represented amplifying T21 lymphocytes for 16 hours using MDA, amplifying T21 lymphocytes for 30 minutes followed by amplifying a half volume of the amplified product for 16 hours using MDA, amplifying YH for 16 hours from the outside to the inside. As can be seen from FIG. 9, if these two kinds of lymphocytes were only subjected to MDA amplification method, since there was an obvious amplification bias, it would result in that the large range of CNV was not able to be recognized from a level of single cell, i.e. it is impossible to find out an obvious difference of genome times in chromosome 21 between the two kinds of cell.

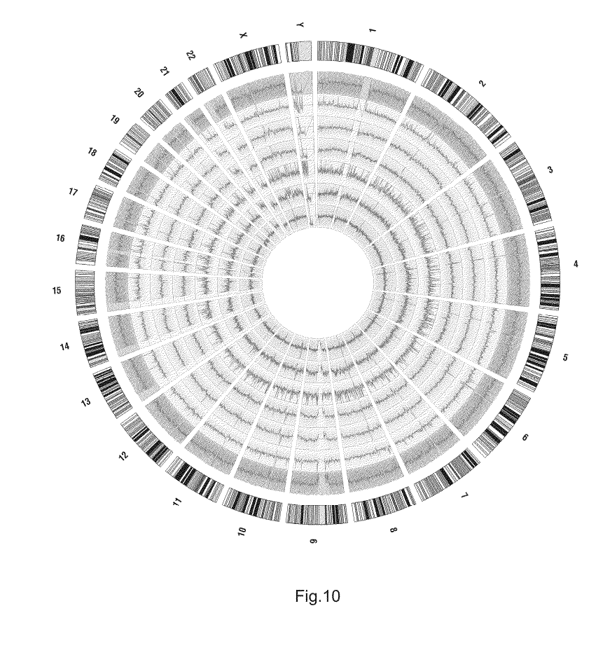

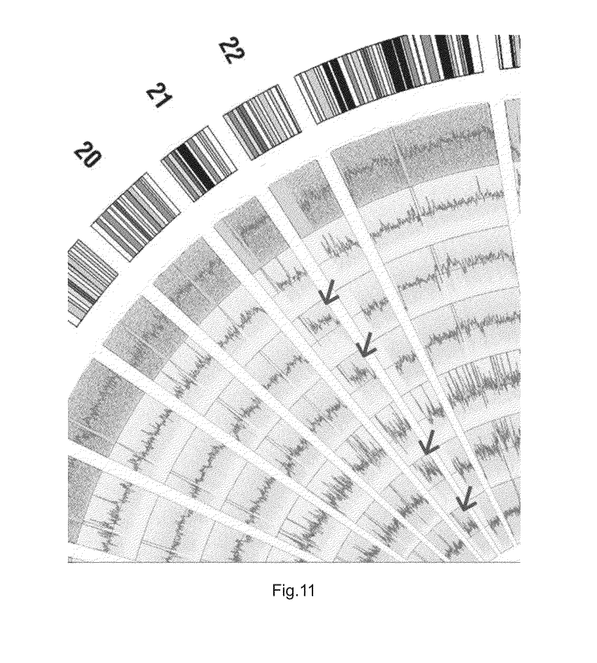

Then, in order to reduce whole genome amplification bias, an experimental proposal that DOP-PCR followed after MDA for different reacting durations was introduced by the inventors of the present disclosure, the single cell was subjected to the whole genome amplification again, which derived from T21 lymphocytes, and the genome Circos images of each sample were obtained according to the above-described method, which were shown in FIG. 10. As shown in FIG. 10, the Circos image showing a detecting result obtained by amplifying T21 lymphocytes using MDA for different durations along with a subsequent DOP-PCR from the outside to the inside, which respectively were 1) DOP-PCR amplification; 2) MDA amplification for 16 hours; 3) MDA amplification for 15 minutes followed by DOP-PCR amplification; 4) MDA amplification for 30 minutes followed by DOP-PCR amplification; 5) MDA amplification for 30 minutes followed by amplifying a half of volume of the amplified product for 16 hours using MDA; 6) MDA amplification for 60 minutes followed by DOP-PCR amplification; 7) MDA amplification for 30 minutes followed by amplifying a half of volume of the amplified product for 16 hours using DOP-PCR. As shown in FIG. 10, for amplifying the single cell derived from T21 lymphocyte, a shifting-up baselines of genome had been found out at a position of chromosome 21 when using MDA for different durations along with a subsequent DOP-PCR, indicating that the number of genome sequences which can be aligned to chromosome 21 was much more than that of the adjacent chromosome. One of the most obvious was: 3) MDA amplification for 15 minutes followed by DOP-PCR amplification; 4) MDA amplification for 30 minutes followed by DOP-PCR amplification; 6) MDA amplification for 60 minutes followed by DOP-PCR amplification; 7) MDA amplification for 30 minutes followed by amplifying a half of volume of the amplified product for 16 hours using DOP-PCR. FIG. 11 was an enlarged view of a partial FIG. 10, i.e. the single cell derived from T21 lymphocyte was amplified using MDA followed by DOP-PCR, a detail view of genome Circos image. It can be seen from FIG. 11, the amplification method of MDA followed by DOP-PCR had an advantage on reducing amplification bias.

Thus, using the method of combining the two kind whole genome amplifications had already been applied, was able to obviously observe that the genome content of chromosome 21 was higher than that of other chromosomes, which could reflect for real that a T21 patient had an extra chromosome at the position of chromosome 21 comparing with that of healthy control.

In the above-described examples, firstly lymphocytic line derived from a donor of the initial Asian genome sequence was used for investigating two amplification methods of MDA and DOP-PCR, demonstrating that the amplification method of the present disclosure could reduce the amplification bias. Then, peripheral blood collected from a female patient having T21 was used; after adding lysis buffer for red blood cells and removing the red blood cells without nuclear, the collected lymphocyte was subjected to a single cell selection by means of mouth-controlled pipette; and the selected single cell was subjected to a whole genome amplifications of DOP, MDA and combination thereof. The amplified product was then subjected to sequencing using a Next-Generation sequencing technology; and the obtained sequencing data was subjected to statistical analyzing, to obtain a distribution of the obtained sequencing in genome of chromosome, so as to determine a difference of genome between a patient having T21 and a healthy individual in a level of single cell. Thereby, it proved that the amplification method of the present disclosure could reduce the amplification bias. And the above described examples demonstrated that a large range of detection of chromosome addition, deletion and transfer in genome could be accomplished by obtaining just a small amount of sample, providing a basis in a level of genome for clinical diagnosis and treatment; on the other hand, the simultaneous detections of SNP and CNV could be achieved with a trace of sample, providing more comprehensive information of genome abnormality, such as tumor cells and etc.

In summary, the new whole genome amplification method of MDA followed by DOP-PCR, not only fills a gap caused by DOP-PCR on low genome amplification coverage and shorter length using MDA, but also reduces non-specific amplification and amplification bias caused by MDA by means of DOP-PCR characteristic of linear amplification, which enable to detect the chromosome addition or deletion in genome in a level of single cell, providing a new idea for reducing amplification bias in whole genome.

### INDUSTRIAL APPLICABILITY

The method of amplifying a whole genome sample, the method for sequencing a whole genome, the method determining whether an abnormal state occurs in a whole genome, the apparatus of amplifying a whole genome sample, the device of sequencing a whole genome, and the system of determining whether an abnormal state occurs in a whole genome of the present disclosure, may effectively subject the whole genome to amplification, sequencing and analyzing, which may reduce amplification bias.

Reference throughout this specification to “an embodiment,” “some embodiments,” “one embodiment”, “another example,” “an example,” “a specific examples,” or “some examples,” means that a particular feature, structure, material, or characteristic described in connection with the embodiment or example is included in at least one embodiment or example of the present disclosure. Thus, the appearances of the phrases such as “in some embodiments,” “in one embodiment”, “in an embodiment”, “in another example, “in an example,” “in a specific examples,” or “in some examples,” in various places throughout this specification are not necessarily referring to the same embodiment or example of the present disclosure. Furthermore, the particular features, structures, materials, or characteristics may be combined in any suitable manner in one or more embodiments or examples.

Although explanatory embodiments have been shown and described, it would be appreciated by those skilled in the art that the above embodiments can not be construed to limit the present disclosure, and changes, alternatives, and modifications can be made in the embodiments without departing from spirit, principles and scope of the present disclosure.

1. A method of amplifying a whole genome sample, comprising:
subjecting the whole genome sample to a first amplification reaction, to obtain a first amplification product;
subjecting the first amplification product to a second amplification reaction, to obtain a second amplification product,
wherein the first amplification reaction is one of a polymerase chain reaction PCR-based amplification reaction and an isothermal amplification reaction, the second amplification reaction is the other of the PCR-based amplification reaction and the isothermal amplification reaction.

2. The method of claim 1, wherein the whole genome sample derives from a whole genome sample of a single cell.

3. The method of claim 1, wherein the first amplification reaction is the isothermal amplification reaction, the second amplification reaction is the PCR-based amplification reaction, the first amplification reaction is at least one selected from a group consisting of strand displacement amplification SDA, multiple displacement amplification (MDA) and rolling circle amplification RCA, the second amplification reaction is at least one selected from a group consisting of linker adapter PCR LA-PCR, degenerate oligonucleotide-primed PCR DOP-PCR, PEP primer extension pre-amplification PCR (PEP-PCR) and LA-PCR.

4. The method of claim 1, wherein the first amplification reaction is MDA, the second amplification reaction is DOP-PCR.

5. The method of claim 1, wherein the first amplification reaction is performed for 15 minutes to 120 minutes.

6. The method of claim 1, further comprising:
constructing a whole genome sequencing-library based on the whole genome amplified product; and
subjecting the whole genome sequencing-library to sequencing.

7. The method of claim 6, further comprising a step of:
extracting the whole genome sample from a single cell.

8. The method of claim 7, wherein the step of extracting the whole genome sample from the single cell further comprises a sub-step of:
isolating the single cell from a biological sample.

9. The method of claim 8, wherein the biological sample is at least one selected from a group consisting of blood, urine, saliva, tissue, germ cell, blastomere and embryo.

10. The method of claim 8, wherein the step of isolating the single cell from the biological sample is performed by at least one selected from a group consisting of dilution, mouth-controlled pipette isolation, micromanipulation, flow cytometry isolation, and microfluidic.

11.-14. (canceled)

15. An apparatus of amplifying a whole genome sample, comprising:
a first amplifying unit, suitable for subjecting the whole genome sample to a first amplification reaction, to obtain a first amplification product;
a second amplifying unit, connected to the first amplifying unit, suitable for subjecting the first amplification product to a second amplification reaction, to obtain a second amplification product,
wherein the first amplifying unit is suitable for performing one of a PCR-based amplification reaction and an isothermal amplification reaction, while the second amplifying unit is suitable for performing the other of the PCR-based amplification reaction and the isothermal amplification reaction.

16. The apparatus of claim 15, wherein the first amplifying unit is suitable for performing an isothermal amplification reaction, while the second amplifying unit is suitable for performing PCR-based amplification reaction.

17. The apparatus of claim 16, wherein the first amplifying unit is suitable for performing MDA, while the second amplifying unit is suitable for performing DOP-PCR.

18. The apparatus of claim 15, further comprising:
a sequencing-library constructing apparatus, connected to the whole genome amplifying apparatus, suitable for constructing a whole genome sequencing-library for a whole genome amplification product; and
a sequencing apparatus, suitable for subjecting the whole genome sequencing-library to sequencing.

19. The apparatus of claim 18, wherein the whole genome amplifying apparatus further comprises:
a single cell isolating unit, for isolating the single cell from a biological sample; and
a single cell lysing unit, for receiving and lysing an isolated single cell, to release the whole genome from the single cell.

20. The apparatus of claim 19, wherein the single cell isolating unit comprises at least one instrument suitable for conducting following operations: dilution, mouth-controlled pipette isolation, micromanipulation, flow cytometry isolation, and microfluidic.

21.-24. (canceled)
